(19) United States
(12) Patent Application Publication
(10) Pub. No.: US 2024/0293042 A1
(43) Pub. Date: Sep. 5, 2024

(54) METHOD FOR REDUCED FALSE POSITIVE ALARMS WHEN DETECTING PATIENT MOVEMENT

(71) Applicant: Murata Vios, Inc., Woodbury, MN (US)

(72) Inventors: Christian ZANDER, Woodbury, MN (US); Atsuhiko HIRATA, Woodbury, MN (US)

(21) Appl. No.: 18/593,450

(22) Filed: Mar. 1, 2024

Related U.S. Application Data
(60) Provisional application No. 63/449,398, filed on Mar. 2, 2023.

(51) Int. Cl. A61B 5/11; A61B 5/00; G16H 40/67

(52) U.S. Cl. CPC A61B 5/1113; A61B 5/0002; A61B 5/0077; A61B 5/1128; A61B 5/746; G16H 40/67; A61B 2562/0219; A61B 2562/0247

(57) ABSTRACT

A patient monitoring system includes a first sensor that monitors patient movement, a second sensor that monitors an area for a presence, and a remote monitoring terminal. An alarm is generated at the remote monitoring terminal when both the first sensor and the second sensor respectively detect the patient movement and the presence.

### BACKGROUND OF THE INVENTION

### 1. Field of the Invention

The present invention relates to a patient monitoring system that detects patient movement and a presence in an area. In particular, the present invention relates to a patient monitoring system that includes a patient monitoring device and that includes a camera and/or weight sensor.

### 2. Description of the Related Art

Home-care patients that require monitoring for fall prevention typically need either a human being to always watch the patient, which can be expensive, or to use a “virtual sitter”. The virtual sitter is typically a sensor that detects movement that may indicate that the patient has left a bed and started to move to another portion of their residence. When the movement is detected, a remote viewer is typically alerted, and the remote viewer can then watch the patient remotely to make sure that no fall has occurred.

However, virtual sitters often cause false alarms when the patient is simply rolling over or sitting up in bed. When these false alarms continuously alert the remote viewer, it can cause an increased expense because the home-care patient is typically charged for each time the remote viewer must watch the patient. Further, when there are a large number of false positives, remote viewers may become complacent, may stop paying full attention, and may not be ready to assist when the home-care patient actually needs assistance.

### SUMMARY OF THE INVENTION

To overcome the problems described above, example embodiments of the present invention provide patient monitoring systems and method of monitoring patients that can detect a patient's movement and a presence in an area to reduce occurrences of false positives.

According to an example embodiment of the present invention, a patient monitoring system includes a first sensing structure or sensor that monitors patient movement, a second sensing structure or sensor that monitors an area for a presence, and a remote monitoring terminal. An alarm is generated at the remote monitoring terminal when both the first sensing structure or sensor and the second sensing structure or sensor respectively detect the patient movement and the presence.

The second sensing structure or sensor can be a camera. The camera can detect motion outside of an exclusion zone configured in a field of view of the camera. The second sensing structure or sensor can include a pressure sensitive structure or sensor.

The first sensing structure or sensor can be configured to be affixed to the patient. The first sensing structure or sensor can include an accelerometer. The first sensing structure or sensor can include a gyroscope.

According to an example embodiment of the present invention, a method of monitoring a patient includes receiving patient-movement data, receiving presence data, and sending an alert based on the patient-movement data and the presence data.

The sending the alert can be based on the patient-movement data indicating that movement of the patient is above a predetermined threshold and based on the presence data indicating either that the patient has left a first area or that someone has entered a second area, which is different from the first area. The first area can be a resting location of the patient, and the second area can be an area adjacent to the resting location of the patient. The patient-movement data can be received from a sensor located on the patient. The method can further include receiving vital-sign data of the patient from the sensor located on the patient.

The presence data can be received from a sensor not located on the patient. The sensor not located on the patient can be a camera. The presence data received from the camera can indicate that motion has been detected outside an exclusion zone configured in a field of view of the camera. The sensor not located on the patient can be a pressure sensor. The alert can be sent to at least one of a bedside monitor, a remote monitoring terminal, or one or more caregivers.

According to example embodiments of the present invention, a patient monitor or a computer include at least one processor that is programmed and/or configured to provide the patent-monitoring method of one of the various other example embodiments of the present invention.

According to a example embodiment of the present invention, a non-transitory computer-readable medium that stores a computer program that, when executed by at least one processor, causes the at least one processor to perform the patent-monitoring method of one of the various other example embodiments of the present invention.

The above and other features, elements, characteristics, steps, and advantages of the present invention will become more apparent from the following detailed description of example embodiments of the present invention with reference to the attached drawings.

### BRIEF DESCRIPTION OF THE DRAWINGS

FIG. 1 shows a patient monitoring system that includes a patient monitoring device, a camera, and a weight sensor.

FIG. 2 shows a top perspective view of a patient monitoring device that can be used in the patient monitoring system shown in FIG. 1.

FIG. 3 shows a bottom perspective view of the patient monitoring device shown in FIG. 2.

FIG. 4 shows a top perspective view of the chest device of the patient monitoring device shown in FIG. 2 with the cables removed.

FIG. 5 shows a bottom perspective view of the battery pack of the patient monitoring device shown in FIG. 2.

FIG. 6 shows a perspective view of internal components of the chest device shown in FIG. 4.

FIG. 7 shows a method of sending an alert based on patient-movement data and presence data.

### DETAILED DESCRIPTION OF EMBODIMENTS

### Example

Patient monitoring systems are typically used for patients who have limited mobility and may be a fall risk. Patient monitoring systems can use sensors or sensing structures to monitor patients to ensure that caregivers are alerted to patient's falls and needs of assistance. These patient monitoring systems are commonly used for home-care patients, but may also be used in hospitals, nursing homes, etc. These patient monitoring systems are very beneficial when it is not possible for a caregiver to personally continually watch the patient. The patient monitoring systems can use two different sensors to monitor patient movement and to monitor a patient presence. For example, a first sensor can monitor patient movement to provide patient-movement data, and a second sensor can monitor an area to provide presence data that indicates motion or presence in area, where presence in an area can include either leaving the area or entering an area. The patient-movement data can be specific to the patient, while the presence data may not be specific to the patient and can be generated by the patient or by someone other than the patient. The first sensor can be attached to the patient to provide patient-specific data and can include an inclinometer, a gyroscope, and/or an accelerometer, and the second sensor is typically not attached to the patient to provide non-specific data and can be a camera and/or a weight sensor. The patient monitoring systems can issue an alert based on the patient-movement data and the presence data.

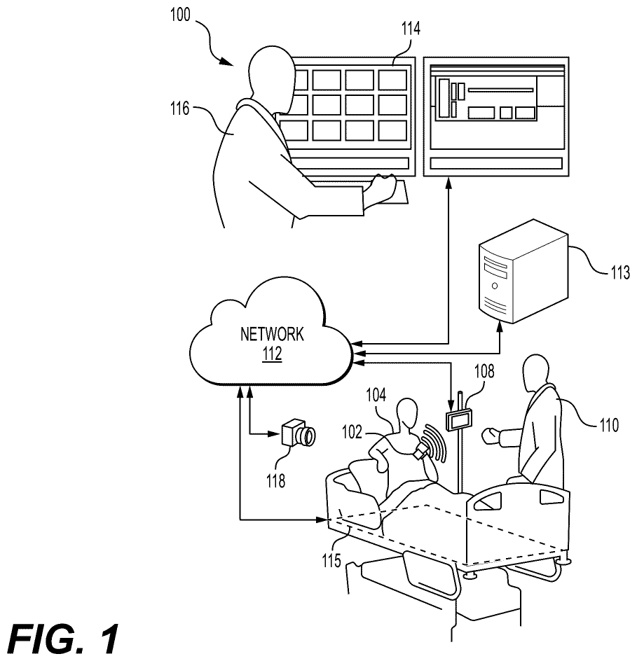

FIG. 1 shows a patient monitoring system 100 which can detect movement of a patient 104 while avoiding false positives. The patient monitoring system 100 can include a patient monitoring device 102, a monitor 108, a weight sensor 115, a camera 118, a network 112, a central server 113, and a remote monitoring terminal 114. The patient monitoring system 100 can include the weight sensor 115, the camera 118, or both the weight sensor 115 and the camera 118. Typically, the patient monitoring system 100 can be located in a hospital, nursing home, private residence, or other suitable location. In the case of a hospital or a nursing home, a caregiver 110 (e.g., a nurse, a doctor, an orderly, a physical therapist, a medical professional, or other caregiver) may sometimes be able to assist or monitor the patient 104 personally, but this may not always be the case. However, in all cases, a remote caregiver 116 (e.g., a nurse, a doctor, an orderly, a physical therapist, a medical professional, or other caregiver) can monitor the patient 104 when the patient monitoring system 100 indicates that the patient 104 has left their position and needs to be monitored in order to watch and make sure that the patient does not suffer a fall.

The network 112 can be a private internal communication network, but could also be connected to the internet if so desired. The network 112 can provide a local area network (LAN) and can use wireless technology and/or cellular communication technology. The network 112 can use both wired and wireless connections. For example, the network 112 can use Wi-Fi to provide a wireless LAN (WLAN). The network 112 can be included in a wide area network (WAN) such as the internet. The network 112 can connect the patient monitoring device 102, the monitor 108, the central server 113, the remote monitoring terminal 114, the weight sensor 115, and the camera 118. The patient monitoring device 102, the monitor 108, the weight sensor 115, and the camera 118 can be connected in a personal area network (PAN). The patient monitoring device 102, the monitor 108, the weight sensor 115, and the camera 118 can be connected using wireless connections or wired connections. For example, one or more Bluetooth, Wi-Fi, or cellular communications can be used to provide wireless connections.

The monitor 108 can be a patient medical monitor which measures and displays, for example, the patient's current medical status including, for example, at least one of oxygen levels, blood pressure, heart rate, etc. For example, the monitor 108 can display an ECG waveform of the patient 104 while also listing any combination of a current blood oxygenation level, blood pressure, hydration level, heart rate, respiration rate, body temperature, blood glucose level, and perspiration of the patient 104. The caregiver 110 can also use the monitor 108 to make notes regarding patient care for the patient 104, to make annotations for vital sign information or other patient information, to send messages to other caregivers, or to log patient activities. The monitor 108 can be mounted on or near the patient's bed and can permit a caregiver 110 to quickly ascertain the patient's condition. The monitor 108 can be connected to the network 112 through either a wired connection or a wireless connection. For example, the monitor 108 can be connected to the network 112 using Wi-Fi and/or cellular communications. A remote caregiver 116 can use a remote monitoring terminal 114 to access the monitor 108 via the network 112. The monitor 108 can be connected to the patient monitoring device 102, the weight sensor 115, and the camera 118 through either a wired connection or a wireless connection. For example, the monitor 108 can include wired connections to the weight sensor 115 and/or the camera 118 and a wireless connection to the patient monitoring device 102, but other connections are also possible.

A processor or processors in the monitor 108 can be configured and/or programmed to determine the specific configuration of the monitor 108 and how to receive, analyze, and display data from the patient monitoring device 102, the weight sensor 115, and the camera 118. Data from the patient monitoring device 102, the weight sensor 115, and the camera 118 can be sent directly to the monitor 108 or can be sent through the network 112 to the monitor 108.

The patient monitoring device 102 can be connected to the patient. The patient monitoring device 102 can include an inclinometer, a gyroscope, and/or an accelerometer which can determine when the patient 104 is moving. The patient monitoring device 102 can be used to determine when the patient 104 is moving farther or faster than predetermined threshold amounts. The patient monitoring device 102 can also be used to validate or to invalidate data from other sensors. For example, if the camera 118 detects motion, but the patient monitoring device 102 does not detect that the movement of the patient or detects that the patient's movement is below a threshold level, then the detected motion can be invalidated and can be attributed to something other than the patient. Alternatively, if the patient monitoring device 102 detects that the movement of the patient above a threshold amount, then the detected motion can be validated and can be attributed to the patient 104. The patient monitoring device 102 can transmit patient-movement data to the monitor 108 and/or the central server 113. The patient monitoring device 102 can transmit the patient-movement data either through the network 112 or directly to the monitor 108.

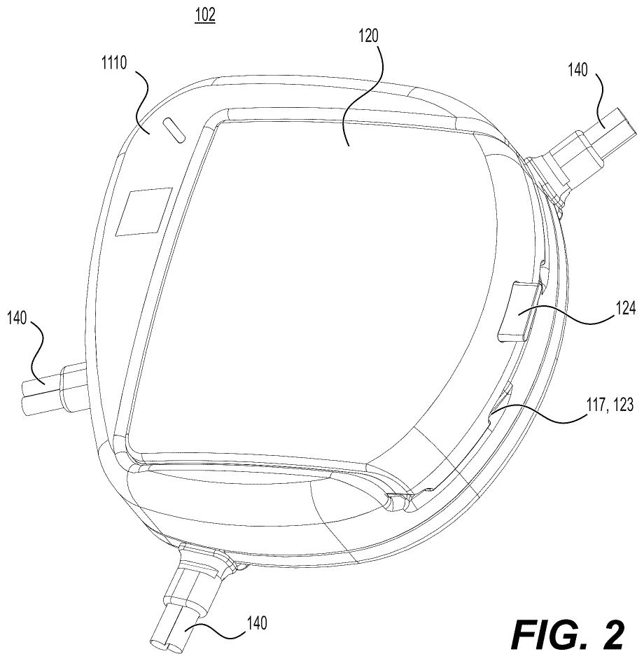

The patient monitoring device 102 can also include adherent electrodes that contact the patient's skin to record various vital signs of the patient, including, for example, heart rhythm (via ECG) and heart rate. The patient monitoring device 102 can also monitor other vital signs, including, for example, any combination of current blood oxygenation level, blood pressure, hydration level, heart rhythm, heart rate, respiration rate, body temperature, blood glucose level, perspiration, and bio-impedance. The patient monitoring device 102 can include a reusable or permanent portion as well as disposable portions. For example, the patient monitoring device 102 can include a permanent housing for storing electrical components that process signals received from and detected in association with the patient and that transmit data to and receive data from other devices, including the monitor 108. The patient monitoring device 102 can include a disposable adherent electrode pad designed to be attached to the patient's chest and can include disposable lead assemblies. The permanent portion of the patient monitoring device 102 can include two or more releasably coupled components that can be detached and swapped out for other components. For example, the permanent portion of the patient monitoring device 102 can include a circuit assembly portion, including for example the chest device 1110 of FIG. 2, that includes processing components and wireless communication components and that is configured to couple with a variety of adapter lead assemblies having various configurations for sensing different sets of patient vital signs. I permanent portion can include a reusable, replaceable portion, such as, for example, the battery pack 120 of FIG. 2.

The patient monitoring device 102 can include circuitry (e.g., one or more processors) that is configured and/or programmed to process and to analyze vital sign information and other information collected from the patient 104. The patient monitoring device 102 can collect raw, pre-processed information which is transmitted to other portions of the patient monitoring system 100 for further processing and analysis. The patient monitoring device 102 can also include circuitry that can process or analyze the raw, pre-processed information and that can then transmit processed data to other portions of the patient monitoring system 100. The patient monitoring device 102 can transmit data to and receive data from the monitor 108, either directly or through the network 112, and can transmit data to and receive data from the central server 113. That is, the patient monitoring device 102 can transmit vital-sign data and patient-movement data to the monitor 108 and/or the central server 113.

Although a single patient monitoring device 102 is shown in FIG. 1, it is possible to use more than one patient monitoring device 102. For example, a first patient monitoring device 108 can be connected to the patient's chest to monitor any combination of current blood oxygenation level, blood pressure, hydration level, heart rate, respiration rate, body temperature, blood glucose level, perspiration, etc., and a second patient monitoring device 108 can be connected to patient's wrist or ankle to monitor movement, posture, etc.

In addition to a first sensor such as the patient monitoring device 102 that monitors a patient's movement, the patient monitoring system 100 can include a second sensor that monitors for a presence in an area. The second sensor can include a weight sensor 115 and/or a camera 118. FIG. 1 shows an arrangement that includes both the weight sensor 115 and the camera 118, but either the weight sensor 115 or the camera 118 can be used alone to monitor for a presence. For example, the weight sensor 115 can monitor if the patient has left a resting location if the weight sensor 115 is under the patient 104, and the camera 118 and the weight sensor 115, if the weight sensor is located near the patient 104, can monitor for the presence of someone in an area near the resting location.

The weight sensor 115 can be connected to the network 112 through either a wired connection or a wireless connection, and the weight sensor 115 can be connected to the patient monitoring device 102 through either a wired connection or a wireless connection. Any suitable weight sensor, pressure sensor, or pressure-sensitive structure can be used as the weight sensor 115. The weight sensor 115 may not be located on or attached to the patient and can be contained in or defined by, for example, a hospital bed, mattress, cushion, chair, mat, etc. The weight sensor 115 can include pressure, stress, proximity, thermal, and/or capacitance sensing element(s) which can be used to determine if the patient 104 is moving out of their resting environment. The weight sensor 115 determines that the patient 104 has left the hospital bed, mattress, cushion, chair, etc. when an output level of the weight sensor 115 exceeds a predetermined threshold amount. As shown In FIG. 1, the weight sensor 115 can be located underneath the patient 104 to determine when a patient leaves an area (e.g., the bed in FIG. 1). Alternatively, the weight sensor 115 can be used to determine when the patient 104 or someone else enters an area. For example, the weight sensor 115 can be located adjacent to the bed so that the weight sensor 115 senses when the patient 104 steps on the weight sensor 115 when the patient 104 leaves the bed. The weight sensor 115 can also be located adjacent the door or the entrance of the patient's room. The weight sensor 115 can used to indicate the presence of someone in a certain area. If the weight sensor 115 is not located under the patient 104, the weight sensor 115 might detect the presence of someone other than the patient. For example, the weight sensor 115 might detect the presence of a caregiver 110 and not the patient. The weight sensor 115 can transmit presence data to the monitor 108 and/or the central server 113, where the presence data can indicate if a patient has left an area (e.g., a resting location or environment of the patient such as a bed or a chair) or has entered an area (e.g., an area immediately adjacent to the resting location or an area nearby the patient including the patient's room). The weight sensor 115 can also be used to validate or to invalidate data from other sensors. For example, if the camera 118 detects motion, but the weight sensor 115 still detects full weight in the patient's bed, then the detected motion can be invalidated and can be attributed to something other than the patient. Alternatively, if the camera 118 detects motion and the weight sensor 115 detects that the patient has left the patient's bed, then the detected motion can be validated and can be attributed to the patient.

The camera 118 can be connected to the network 112 through either a wired connection or a wireless connection. The camera 118 may not be located on or attached to the patient. The camera 118 can be connected to the patient monitoring device 102 through either a wired connection or a wireless connection. Any vision system that can detect motion can be used as the camera 118. The camera 118 can include a microphone and a speaker such that the remote caregiver 116 can communicate with the patient 104 through the camera 118. The camera 118 can be physically attached or mounted to the monitor 108. Alternatively, the camera 118 can be integrated with the monitor 108.

The camera 118 can be a remotely controllable closed-circuit camera or other suitable camera which can be connected to the remote monitoring terminal 114 through the network 112. The camera 118 can sense when the patient 104 is or has moved out of the resting environment and can send an indication to the remote monitoring terminal 114 through the network 112. The camera 118 can transmit presence data to the monitor 108 and/or the central server 113, where the presence data indicates if the patient leaves an area or enters an area. The camera 118 can be used to detect motion. Simple image processing can be used to provide motion detection without providing facial recognition. That is, motion or presence can be detected without specifically determining that the patient is moving or present in an area. For example, the camera 118 might detect the motion of a caregiver 110 and not the patient 104. The camera 118 can be operated so that any motion in the resting environment of the patient 104 is excluded from determining whether the patient 104 is moving out of the resting environment, i.e., an exclusion zone can be created. By excluding the resting environment of the patient 104, it is possible to prevent “false positive” readings for when the patient 104 is merely readjusting or stretching.

The remote monitoring terminal 114 can be located in the same building as the patient 104 or can be located in a different building from the patient 102. For example, in a home setting, the remote monitoring terminal 114 can be remotely located from the patient 104. Alternatively, in a hospital setting, the remote monitoring terminal 114 can be located at a nurse station on the same floor as the patient 102. The remote monitoring terminal 114 can allow a single remote caregiver to monitor multiple patients at the same time. The remote monitoring terminal 114 can communicate with the camera 118 through the network 112.

The central server 113 can be connected to the remote monitoring terminal 114 directly and/or through the network 112. The central server 113 can be located with the remote monitoring terminal 114, e.g., in the same room, or can be located remotely from the remote monitoring terminal 114, e.g., in the cloud or in a different building. Any suitable computer can be used as the central server 113. The central server 113 can continuously record in a database all of the data received from the patient monitoring device 102, the monitor 108, the weight sensor 115, and the camera 118 to compile a historical record of the patient's location, movement, and medical status. This historical record is preserved for recordkeeping purposes, and to provide the caregiver 110 and the remote caregiver 116 with a resource to review in the case that there is a question as to the patient's condition at certain times.

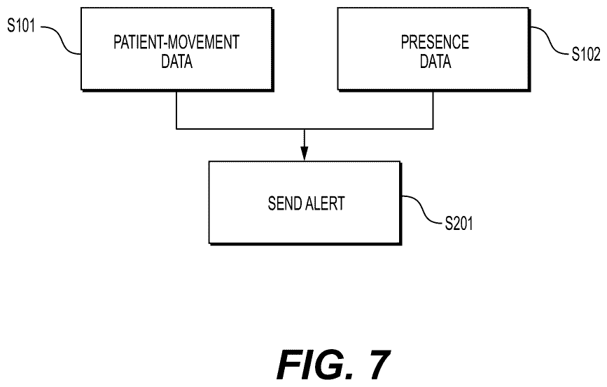

The patient monitoring system 100 can send an alert when it is detected that the patient 104 is leaving their resting environment. FIG. 7 shows a method of sending an alert that includes step S101 of receiving patient-movement data and step S102 of receiving presence data and then, in step S201, sending an alert based on patient-movement data and presence data. Steps S101 and S102 can occur simultaneously or alternatively. Step S101 can occur first, or step S102 can occur first. The alert is triggered when patient-movement data from the patient monitoring device 102 exceeds a predetermined threshold and presence data from at least one of the weight sensor 115 and the camera 118 indicates that the patient has left an area or someone has entered an area. That is, when both the patient monitoring device 102 and the weight sensor 115, or when both the patient monitoring device 102 and the camera 118, indicate that the patient 104 is more than likely leaving their resting environment, an alarm is triggered. The alert can indicate that the patient should be actively monitored.

The alert can be sent to any combination of (1) a caregiver or a group of caregivers, (2) the remote monitoring terminal 114, and (3) the monitor 108. The alert can be sent as, for example, an email, text message, or page to the caregiver or group of caregivers. The alert can be sent to the remote monitoring terminal 114 and/or monitor 108 as a visual alert and/or an auditory alert.

The alert can be sent by the monitor 108 or by the central server 113. The monitor 108 can include one or more processors that can be programmed and/or configured to send the alert based on the patient-movement data and the presence data. And the central server 113 can include one or more processors that can be programmed and/or configured to send the alert based on the patient-movement data and the presence data. If the alert is sent by the monitor 108, then the monitor 108 can generate a visual alert and/or an auditory alert to alert any caretakers that not in the room with the patient but are nearby.

By requiring both the patient monitoring device 102 and at least one of the weight sensor 115 and the camera 118 to indicate that the patient 104 is more than likely leaving their resting environment before triggering the alarm, it is possible to avoid or to reduce the occurrence of any “false positive” alarms which would waste the resources of a remote caregiver 116. Accordingly, every time the remote caregiver 116 receives an alarm, they can be more certain that there is a real need for them to monitor the movement of the patient 104 to watch for falls or other emergencies.

If only the camera 118 is used to determine when to trigger an alarm, a “false positive” could happen when the caregiver 110 enters the field of view of the camera 118. If only the weight sensor 115 was used to determine when to trigger an alarm, a “false positive” would happen when the patient readjusts their weight in the resting environment. If only the patient monitoring device 102 was used to determine when to trigger an alarm, a “false positive” could happen when the patient moves or turns over in the resting environment.

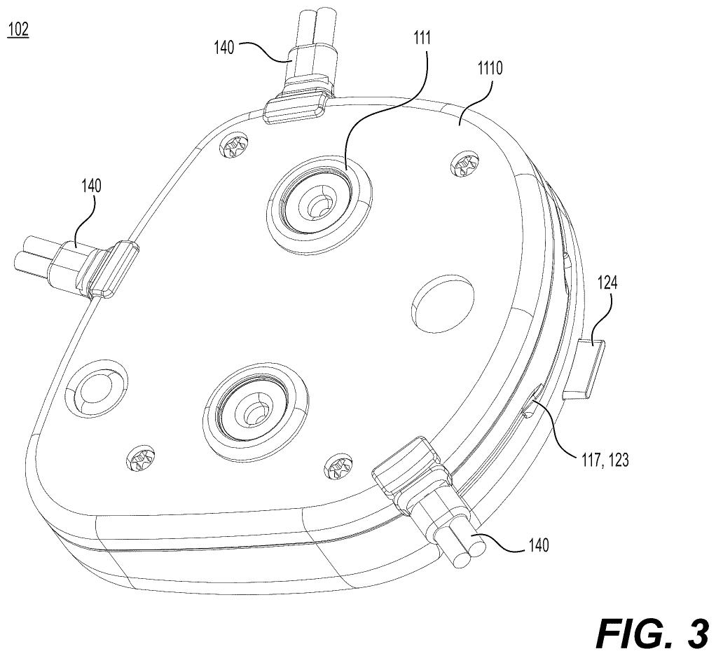

FIGS. 2 and 3 show a patient monitoring device 102. In the example of patient monitoring device 102 shown in FIGS. 2 and 3, a chest device 1110 of the patient monitoring device 102 can be attached to the patient's chest. However, the patient monitoring device 102 can be attached to one or more other areas on the patient's body. The patient monitoring device 102 can detect, record, store, and transmit various vital signs and other information of the patient. For example, the patient monitoring device 102 can include adherent electrodes that contact the patient's skin to measure various biological information, vital signs, and patient information including, but not limited to, any combination of heart rhythm, heart rate, blood pressure, body temperature, respiration rate, blood oxygenation, blood glucose level, hydration levels, perspiration, and bio-impedance. The patient monitoring device 102 can also track patient movement, activity, posture, etc.

The patient monitoring device 102 can also communicate with one or more other computing devices, either through wired or wireless communication. For example, the patient monitoring device 102 can use one or more of Bluetooth, Wi-Fi, or cellular communications to communicate with other computing devices such as monitors, personal computers, tablet devices, mobile phones, central servers, or a cloud-based network. As an example, the patient monitoring device 102 can transmit vital-sign information collected from the patient to a tablet device or a personal computer that operates as a bedside monitor. The tablet or personal computer can process received information and display the information in a readily understandable format to a caretaker or other user. For example, a tablet device, which, for example, could be the monitor 108, can receive vital-sign information from the patient monitoring device 102 through a Bluetooth and/or Wi-Fi connection and display an electrocardiogram (ECG) waveform of the patient, as well as information on any combination of the patient's heart rate, respiration rate, blood oxygenation level, body temperature, and/or other vital signs. As another example, the patient monitoring device 102 can be periodically connected to a computing device (such as monitor 108) through a wired connection to allow information collected by the patient monitoring device 102 to be stored, processed, and displayed by the computing device and/or transferred to one or more other computing devices (e.g., personal computers, servers located at the hospital, cloud storage servers, etc.).

Furthermore, information recorded by the patient monitoring device 102 can be transmitted to other computing devices to provide real-time or near real-time analysis of the patient's condition, and to provide tracking of vital-sign information of the patient over time. For example, the information recorded by the patient monitoring device 102 can be transmitted to a display device to allow caregivers to observe the information and adjust patient care based on the information. The information can also be transmitted to a central information repository to log and store historical vital-sign and other information of the patient. Both real-time and historical vital-sign information, and other information, of a patient can be accessed by caregivers who are not at the same physical location as the patient. For example, vital-sign information collected by the patient monitoring device 102 can be transmitted to a mobile device owned by the patient or caregiver (e.g., a smart phone) to allow the patient or caregiver to view the information. The information can further be transmitted to a central server that can be accessed by one or more caregivers (e.g., using personal computers and/or mobile devices) to allow the caregivers to view the collected information and make patient care decisions for the patient from a location that is remote from where the patient is located.

Other components that can be included as part of the patient monitoring device 102 include a power supply, buttons, or other input mechanisms for receiving user input, one or more audible alarms or speakers, and lights or a display screen. The patient monitoring device 102 can further include input mechanisms such as, for example, buttons, keys, or a touch screen. The input mechanisms can allow the patient or a caregiver to adjust settings for the patient monitoring device 102, perform various tests (for example, sensor tests, battery power level tests, etc.), or reset one or more alarms for the patient monitoring device 102. The input mechanisms can also allow the patient to place a distress call (e.g., to a caregiver or to a hospital alert system) if the patient needs assistance.

As shown in FIGS. 2 and 3, the patient monitoring device 102 can include a chest device 1110 with snaps 111 that can be connected to adherent electrodes to connect the patient monitoring device 102 to a patient, a battery pack 120 to provide power to the chest device 1110, and cables 140 each connected to different sides of the chest device 1110 that attach signal leads to the chest device 1110. The cables 140 can include, for example, ECG cables and pulse ox cables. The battery pack 120 can include standard disposable batteries or a rechargeable battery, and the battery pack 120 is removable such that it can be replaced with a different battery pack.

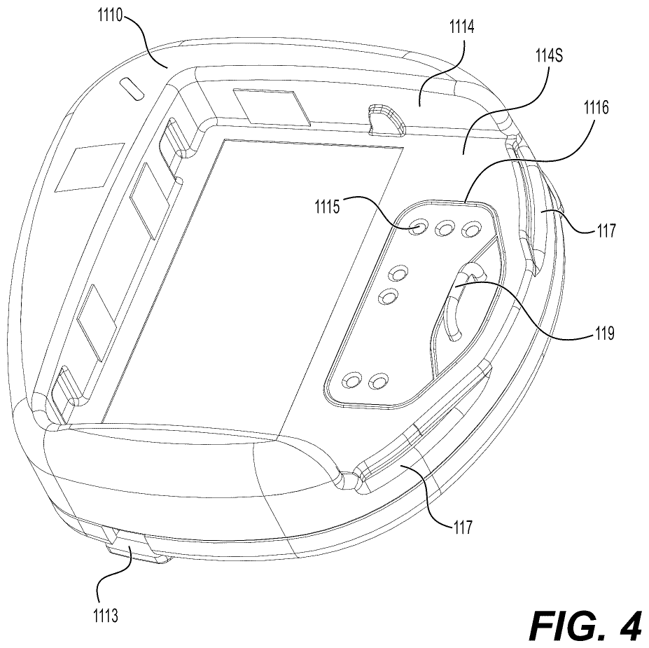

FIG. 4 shows that the chest device 1110 includes a recess 1114 to receive the battery pack 120. The battery pack 120 is removeable from the chest device 1110, i.e., the battery pack 120 can be connected to and disconnected from the chest device 1110. The recess 1114 defines a recessed surface 114S of the chest device 1110, and the recessed surface 114S can be flat or substantially flat so that the recessed surface 114S can be easily cleaned by a user.

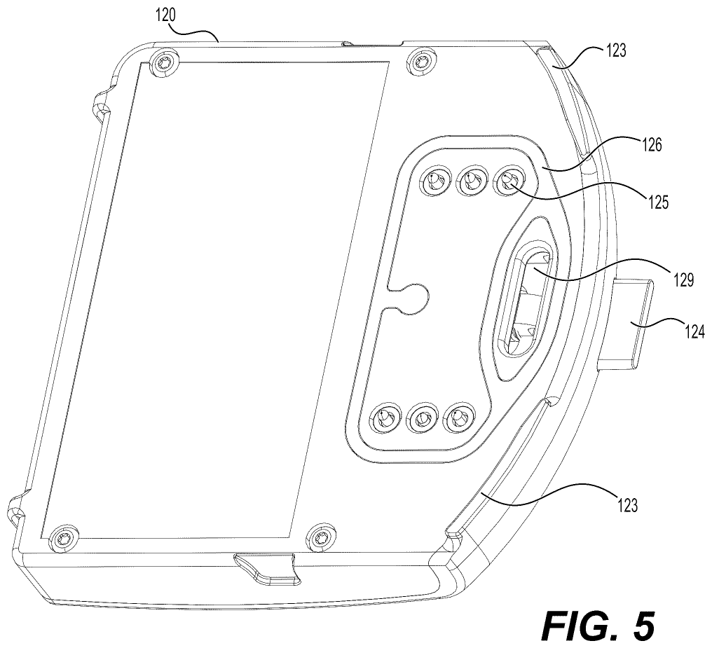

FIG. 5 shows a bottom perspective view of the battery pack 120. The battery pack 120 makes electrical connection with the chest device 1110 via a connector system including chest device connectors 1115 and battery pack connectors 125, which are electrical connectors that are described in more detail below.

External features of the chest device 1110 are described with respect to FIGS. 2-4. The chest device 1110 can be made from a clam-shell type construction in which a top housing is attached to a bottom housing, the interior of which houses electronic circuitry to perform patient monitoring and communication operations. FIG. 4 shows that the chest device 1110 can include openings 1113 through which the cables 140 can be attached to the chest device 1110. The openings 1113 can all have identical shapes or can have different shapes. If a cable or component is not needed, then a plug (not shown) can be inserted in the corresponding opening 1113. The plug can be a permanent plug inserted during a manufacturing or assembly process of the chest device 1110 or can be a removable plug that can be inserted or removed as needed. For example, a customized chest device 1110 with a customized cable combination can be made by replacing an unwanted cable with a plug to close the opening 1113 during the manufacturing or assembly process. If the openings 1113 have identical shapes, then different shaped plugs do not have to be prepared in the manufacturing or assembly process.

As shown in FIGS. 4 and 5, the chest device 1110 and the battery pack 120 include respective electrical connections that are able to be mated and unmated. In particular, the chest device 1110 includes chest device connectors 1115 provided in recess 1114, and the battery pack 120 includes battery pack connectors 125. The chest device connectors 1115 can be electrical targets (for example, pogo targets), and the battery pack connectors 125 can be electrical pins (for example, movable pins such as pogo pins). The battery pack connectors 125 can be movable pins that have a direction of motion perpendicular to the recessed surface 114S of the chest device 1110 when the battery pack 120 is mated to the chest device 1110.

The arrangement and location of the chest device connectors 1115 and battery pack connectors 125 are not limited to the specific arrangement and location of chest device connectors 1115 and battery pack connectors 125 shown in FIGS. 4 and 5. As an example, the chest device 1110 may be provided with pogo pins and the battery pack 120 may be provided with pogo targets. However, the chest device 1110 can be designed to have a longer life cycle than the battery pack 120, and thus the chest device connectors 1115 can be pogo targets since pogo targets generally have longer life cycles than pogo pins.

The chest device 1110 can include chest device connectors 1115 that do not mate with battery pack connectors 125 of the battery pack 120. For example, one or more of the chest device connectors 1115 can be a data connection for a computer or similar device to perform debugging, maintenance, and the like on the chest device 1110. Similarly, the battery pack 120 can include battery pack connectors 125 that do not mate with chest device connectors 1115 of the chest device 1110. For example, one or more of the battery pack connectors 125 can be a data connection to provide temperature data from a thermistor while a battery of the battery pack 120 is being charged.

For example, the chest device connectors 1115 can be separated from one another by at least about 2 mm within manufacturing and/or measurement tolerances, and the battery pack connectors 125 can be separated from one another by at least about 2 mm within manufacturing and/or measurement tolerances. Accordingly, corrosion of the chest device connectors 1115 and the battery pack connectors 125 is further able to be significantly reduced or prevented even if water or other fluids intrude upon the chest device connectors 1115 and the battery pack connectors 125.

The chest device connectors 1115 and the battery pack connectors 125 each can include a metal or a metal coating that is resistant to corrosion. For example, the chest device connectors 1115 and the battery pack connectors 125 can include stainless steel or can be plated by nickel or gold.

As shown in FIGS. 2-5, the battery pack 120 can include protrusions or lips 123 that are received by corresponding indents 117 of the chest device 1110. The protrusions 123 each can extend higher than an uppermost portion of each of the battery pack connectors 125, with respect to the orientation shown in FIG. 5. Accordingly, when the battery pack 120 is separated from the chest device 1110, the battery pack connectors 125 are unlikely to be scratched or corroded, for example, by a user placing the battery pack 120 on a desk surface with the battery pack connectors 125 facing the desk surface.

As shown in FIG. 4, the chest device 1110 includes a ridge 1116 that surrounds the chest device connectors 1115, and as shown in FIG. 5, the battery pack 120 includes a gasket 126 that surrounds the battery pack connectors 125. The ridge 1116 and the gasket 126 can have corresponding and similar shapes. However, the gasket 126 can be wider than the ridge 1116 to ensure that the ridge 1116 is fully engaged and surrounded by the gasket 126. The gasket 126 can include additional material (e.g., protrusions) due to an injection molding process used to form the gasket 126, and/or to help secure the gasket 126 to the battery pack 120.

A lock structure of the chest device 1110 and the battery pack 120 is described below with respect to FIGS. 4 and 5. As shown in FIGS. 4 and 5, the chest device 1110 includes a loop 119 that is received by an opening 129 in the battery pack 120. A user can press the button 124 to disengage the loop 119, thereby enabling the battery pack 120 to be unmated from the chest device 1110.

The chest device 1110 does not need to be disconnected or discarded to change or modify the battery pack 120. With a removable battery pack 120, the chest device 1110 can remain attached to the patient and does not need to be removed from the electrodes connected to the cables 140. If rechargeable, the battery pack 120, when depleted, can be replaced with a charged battery pack while the chest device 1110 remains attached to the patient. If the battery charge of the battery pack 120 reduces too much or the batteries deteriorate after many times of charging and discharging, a user can simply replace the old battery pack 120 with a new one, without having to replace the chest device 1110 that is relatively more expensive than the battery pack 120 because of the circuitry included in the chest device 1110.

A battery of the battery pack 120 can be either a replaceable battery or a rechargeable battery. If the battery pack 120 includes a rechargeable battery, then the battery pack 120 can be charged using a charging station. As an example, the battery of the battery pack 120 can be a lithium ion battery that has a voltage between about 4.0 V and 4.2 V. The battery pack 120 can be properly oriented with respect to the chest device 1110 according to the lock structure including the loop 119 and the opening 129, and according to the mating structure of the indents 117 and the protrusions 123.

Although the circuitry components of the battery pack 120 can include circuitry components that are not related to battery functions (e.g., functions related to the chest device 1110), the battery pack 120 can only include circuitry components related to battery functions (e.g., charging, battery status, short circuit protection, and the like). Optionally, the power and electrical signals can be routed by discrete wires or another suitable mechanism. According to the arrangement shown in FIGS. 2-5, the battery pack 120 can change without affecting the chest device 1110. That is, the battery pack 120 can vary in thickness to accommodate a different battery that can have a longer life or be made with a different battery technology without having to redesign or reconfigure the chest device 1110.

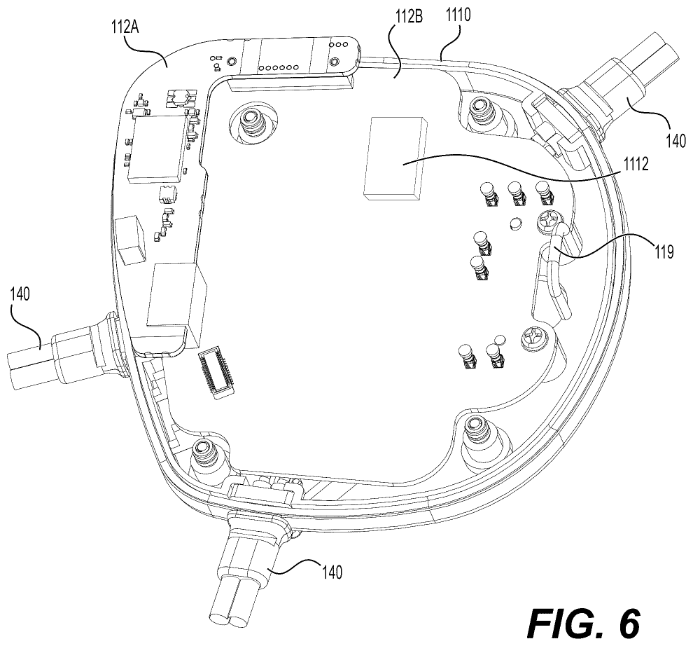

As shown in FIG. 6, the chest device 1110 can include a first PCB 112A and a second PCB 112B can include mounted circuitry components. For example, at least an inclinometer, a gyroscope, and/or an accelerometer 1112 can be located on the second PCB 112B as shown in FIG. 6 or on the first PCB 112A. The inclinometer can be a single-axis type or a multiple-axis type, the gyroscope can be a single-axis type or a multiple-axis type, and the accelerometer can be a single-axis type or a multiple-axis type. The inclinometer, the accelerometer, and the gyroscope can be combined in a single sensor. In addition, the first PCB 112A and the second PCB 112B can be rigid and made from any suitable material. However, any suitable substrate can be used instead of the first PCB 112A and the second PCB 112B.

Suitable processors for the execution of a computer program include, by way of example, both general and special purpose microprocessors, and the sole processor or one of multiple processors of any kind of computer. Generally, a processor will receive instructions and data from a read-only memory or a random-access memory or both. Generally, a computer will also include, or be operatively coupled to communicate with, one or more mass storage devices for storing data files, such mass storage devices include magnetic disks, such as internal hard disks and removable disks, magneto-optical disks, and optical disks. Non-transitory storage devices that can store computer programs and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application specific integrated circuits). To provide for interaction with a user, the features can be implemented on a computer having a display device such as a CRT (cathode ray tube) or LCD (liquid crystal display) monitor to display information to the user and a keyboard and a pointing device such as a mouse or a trackball by which the user can provide input to the computer.

The features, functions, and methods discussed above can be implemented in a computer system that includes a back-end component, such as a data server, or that includes a middleware component, such as an application server or an internet server, or that includes a front-end component, such as a client computer having a graphical user interface or an internet browser, or any combination of them. The components of the computer system can be connected by any form or medium of digital data communication such as a communication network. Examples of communication networks include, e.g., a LAN, a WAN, and the computers and networks forming the internet. The computer system can include clients and servers. A client and server are generally remote from each other and typically interact through a network, such as the networks described above. The relationship of client and server arises by virtue of computer programs running on the respective computers and having a client-server relationship to each other.

Computer-readable media that stores a computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1: A patient monitoring system comprising:
a first sensor that monitors patient movement;
a second sensor that monitors an area for a presence; and
a remote monitoring terminal; wherein
an alarm is generated at the remote monitoring terminal when both the first sensor and the second sensor respectively detect the patient movement and the presence.

2: The patient monitoring system according to claim 1, wherein the second sensor is a camera.

3: The patient monitoring system according to claim 2, wherein the camera detects motion outside of an exclusion zone configured in a field of view of the camera.

4: The patient monitoring system according to claim 1, wherein the second sensor includes a pressure sensitive sensor.

5: The patient monitoring system according to claim 1, wherein the first sensor is configured to be affixed to the patient.

6: The patient monitoring system according to claim 1, wherein the first sensor includes an accelerometer.

7: The patient monitoring system according to claim 1, wherein the first sensor includes a gyroscope.

8: A method of monitoring a patient comprising:
receiving patient-movement data;
receiving presence data; and
sending an alert based on the patient-movement data and the presence data.

9: The method of claim 8, wherein the sending the alert is based on the patient-movement data indicating that movement of the patient is above a predetermined threshold and based on the presence data indicating either that the patient has left a first area or that someone has entered a second area, which is different from the first area.

10: The method of claim 9, wherein the first area is a resting location of the patient, and the second area is an area adjacent to the resting location of the patient.

11: The method of claim 8, wherein the patient-movement data is received from a sensor located on the patient.

12: The method of claim 11, further comprising receiving vital-sign data of the patient from the sensor located on the patient.

13: The method of claim 8, wherein the presence data is received from a sensor not located on the patient.

14: The method of claim 13, wherein the sensor not located on the patient is a camera.

15: The method of claim 14, wherein the presence data received from the camera indicates that motion has been detected outside an exclusion zone configured in a field of view of the camera.

16: The method of claim 13, wherein the sensor not located on the patient is a pressure sensor.

17: The method of claim 8, wherein the alert is sent to at least one of a bedside monitor, a remote monitoring terminal, or one or more caregivers.

18: A patient monitor comprising at least one processor that is programmed and/or configured to provide the patent-monitoring method of claim 8.

19: A computer comprising at least one processor that is programmed and/or configured to provide the patent-monitoring method of claim 8.

20: A non-transitory computer-readable medium that stores a computer program that, when executed by at least one processor, causes the at least one processor to perform the patent-monitoring method of claim 8.
